(19) United States
(12) Patent Application Publication
(10) Pub. No.: US 2017/0239696 A1
(43) Pub. Date: Aug. 24, 2017

(54) Medical Instrument with Instrument Flushing System

(71) Applicant: Arthrex, Inc., Naples, FL (US)

(72) Inventor: Robert McGregor Weber, Chino Hills, CA (US)

(73) Assignee: Arthrex, Inc., Naples, FL (US)

(21) Appl. No.: 15/048,682

(22) Filed: Feb. 19, 2016

(51) Int. Cl. B08B 9/027; A61B 17/00

(52) U.S. Cl. CPC B08B 9/027; A61B 17/00234; A61B 2017/00853

(57) ABSTRACT

A medical instrument with an elongated outer housing and inner drive shaft and including an instrument flushing system that enables the medical instrument to be effectively cleaned after use is disclosed. The medical instrument may be configured such that the instrument flushing system receives a flushing fluid through a port at a proximal end of the instrument and directs the flushing fluid through a flushing fluid plenum created between an inner surface of the elongated outer housing and an outer surface of the inner drive shaft. The flushing fluid plenum may be maintained by a standoff formed from a collar with one or more axially extending grooves. The instrument flushing system may include a seal to seal portions of a drive system in communication with the inner drive shaft and extending radially outward from a main body of the medical instrument.

### FIELD OF THE DISCLOSURE

The present disclosure generally relates to medical instruments, and more particularly to medical instruments with flushing systems for cleaning the instruments after use.

### BACKGROUND

Medical instruments used for surgical procedures are commonly flushed after use to remove any bodily tissue that may have accumulated within the instrument during use. The medical instruments may be disassembled to clean and remove the bodily tissue. Some medical instruments include flush systems to flush internal aspects of the medical instruments. However, many of these systems do not flush all tissue an blood from interior aspects of the devices. Thus, there remains a need for an efficient system for flushing internal aspects of medical instruments.

### SUMMARY

A medical instrument with an elongated outer housing and inner drive shaft and including an instrument flushing system that enables the medical instrument to be effectively cleaned after use is disclosed. The medical instrument may be configured such that the instrument flushing system receives a flushing fluid through a port at a proximal end of the instrument and directs the flushing fluid through a flushing fluid plenum created between an inner surface of the elongated outer housing and an outer surface of the inner drive shaft. The flushing fluid plenum may be maintained by a standoff formed from a collar with one or more axially extending grooves. The instrument flushing system may include a seal configured to seal portions of a drive system in communication with the inner drive shaft and extending radially outward from a main body of the medical instrument.

The medical instrument may be formed from an elongated outer housing with a hollow inner plenum, whereby a distal end of the elongated outer housing includes an outer shaft distal opening. The medical instrument may also include a main body extending proximally from a proximal end of the elongated outer housing. The medical instrument may include an inner drive shaft positioned within the elongated outer housing and extending into a hollow cavity within the main body, whereby the inner drive shaft may be sized such that a flushing fluid plenum exists in the hollow inner plenum of the elongated outer housing between an outer surface of the inner drive shaft and an inner surface of the elongated outer housing. The medical instrument may also include a drive system in communication with the inner drive shaft and extending radially outward from the main body, whereby the drive system may be configured to move the inner drive shaft axially within the hollow inner plenum of the elongated outer housing to drive distally positioned working heads. The medical instrument may also include an instrument flushing system in communication with the hollow cavity within the main body and configured to pass flushing fluid through the hollow cavity within the main body and through the flushing fluid plenum in the hollow inner plenum of the elongated outer housing between the outer surface of the inner drive shaft and the inner surface of the elongated outer housing.

A method of cleaning a medical instrument may include providing fluid to the medical instrument formed from an elongated outer housing with a hollow inner plenum, whereby a distal end of the elongated outer housing includes an outer shaft distal opening. The medical instrument may include a main body extending proximally from a proximal end of the elongated outer housing and an inner drive shaft positioned within the elongated outer housing and extending into a hollow cavity within the main body, whereby the inner drive shaft may be sized such that a flushing fluid plenum exists in the hollow inner plenum of the elongated outer housing between an outer surface of the inner drive shaft and an inner surface of the elongated outer housing. The medical instrument may include a drive system in communication with the inner drive shaft and extending radially outward from the main body, whereby the drive system may be configured to move the inner drive shaft axially within the hollow inner plenum of the elongated outer housing. The medical instrument may include an instrument flushing system in communication with the hollow cavity within the main body and configured to pass flushing fluid through the hollow cavity within the main body and through the flushing fluid plenum in the hollow inner plenum of the elongated outer housing between the outer surface of the inner drive shaft and the inner surface of the elongated outer housing. The method may also include enabling flush fluid to flow into the instrument flushing system.

An advantage of the medical instrument is that the instrument flushing system enables tissue particles and blood to be easily and effectively flushed from internal aspects of the medical instrument such as within the elongated outer housing.

Another advantage of the medical instrument is the standoff of the instrument flushing system within the elongated outer housing positions the inner drive shaft within the elongated outer housing while enabling flushing fluid to flow past the standoff by flowing through the axially extending grooves of the standoff.

Yet another advantage of the medical instrument is the seal surrounding the first drive handle protruding from the main body of the medical instrument eliminates or substantially eliminates leakage of the flushing fluid form the instrument flushing system.

Another advantage of the medical instrument is one or more ports in the instrument flushing system that may be configured to be releasably coupled to a flushing fluid supply source. The port may also be aligned axially with the hollow cavity in the main body and with the flushing fluid plenum in the elongated outer housing.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a side view of a medical instrument including an instrument flushing system.

FIG. 2 is a perspective view of the medical instrument of FIG. 1.

FIG. 3 is an exploded perspective view of the medical instrument of FIG. 1.

FIG. 4 is yet another exploded perspective view of the medical instrument of FIG. 1 with a portion of the main body removed, exposing the hollow cavity within the main body.

FIG. 5 is a perspective view of the inner drive shaft positioned within the elongated outer housing via a standoff having a plurality of ridges that extend outwardly in a radial direction.

FIG. 6 is a perspective view of a portion of a seal used to seal a first drive handle extending from the main body.

FIG. 7 is a flow diagram of a method of using the medical instrument including an instrument flushing system of FIG. 1.

FIG. 8 is a perspective view of the inner drive shaft positioned within the elongated outer housing via a standoff having a plurality of ridges that extend inwardly in a radial direction.

FIG. 9 is an end view of the standoff shown in FIG. 8.

FIG. 10 is a perspective view of the inner drive shaft positioned within the elongated outer housing via a standoff having a plurality of ridges that extend inwardly in a radial direction and.

FIG. 11 is an end view of the standoff shown in FIG. 10.

FIG. 12 is an end view of a standoff without a plurality of ridges.

### DETAILED DESCRIPTION

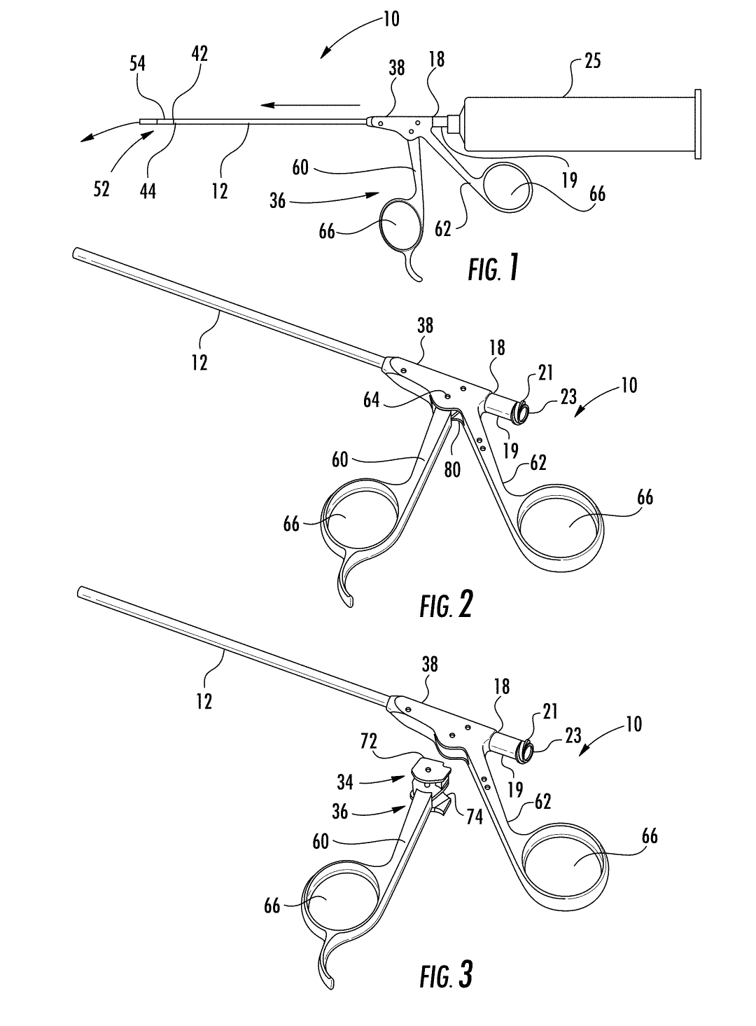

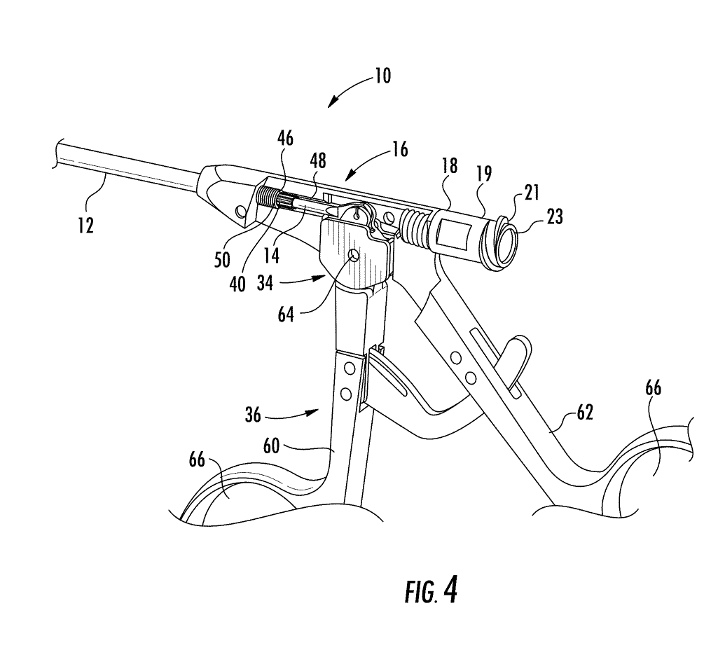

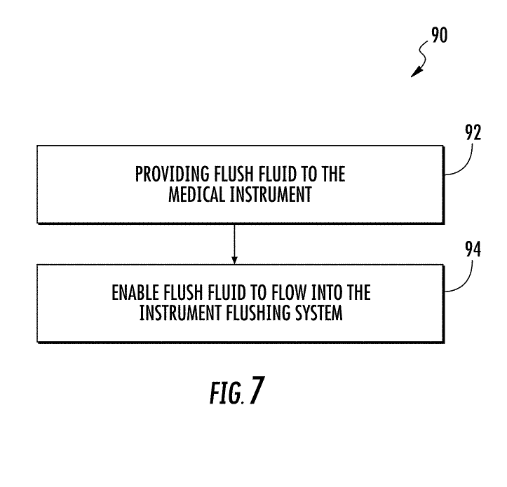

As shown in FIGS. 1-12, a medical instrument 10 with an elongated outer housing 12 and inner drive shaft 14 and including an instrument flushing system 16 that enables the medical instrument 10 to be effectively cleaned after use is disclosed. The medical instrument 10 may be configured such that the instrument flushing system 16 receives a flushing fluid through a port 18 at a proximal end 20 of the instrument 10 and directs the flushing fluid through a flushing fluid plenum 22 created between an inner surface 24 of the elongated outer housing 12 and an outer surface 26 of the inner drive shaft 14. The flushing fluid plenum 22 may be maintained by a standoff 28 formed from a collar 30 with one or more axially extending grooves 32. The instrument flushing system 16 may include a seal 34 to seal portions of a drive system 36 in communication with the inner drive shaft 14 and extending radially outward from a main body 38 of the medical instrument 10.

The medical instrument 10 may include an elongated outer housing 12 with a hollow inner plenum 40. A distal end 42 of the elongated outer housing 12 may include an outer shaft distal opening 44. A main body 38 may be coupled to a proximal end of the elongated outer housing 12. The main body 38 may extend proximally from a proximal end 46 of the elongated outer housing 12. The main body 38 may be formed from any appropriate material, such as, but not limited to, plastic and stainless steel. The main body 38 may include a hollow cavity 48 positioned therein and having any appropriate configuration. The proximal end 46 of the elongated outer housing 12 may extend into a distal end 50 of the hollow cavity 48.

An inner drive shaft 14 may be positioned within the elongated outer housing 12 and may extend into the hollow cavity 48 within the main body 38. The inner drive shaft 14 may be sized such that a flushing fluid plenum 22 exists in the hollow inner plenum 40 of the elongated outer housing 12 between an outer surface 26 of the inner drive shaft 14 and an inner surface 24 of the elongated outer housing 12 when the inner drive shaft 14 is positioned within the outer housing 12. The inner drive shaft 14 may be formed form any appropriate material, such as, but not limited to, plastic and stainless steel. The inner drive shaft 14 may have a generally cylindrical cross-sectional shape or other appropriate shape. As shown in FIG. 1, the inner drive shaft 14 may include a one or more working heads 52 attached to a distal end 54 of the inner drive shaft 14. The working head 52 may be, but is not limited to being, one or more set of jaws, convention working heads or yet to be developed working heads.

The medical instrument 10 may include a drive system 36, as shown in FIGS. 1-4, in communication with the inner drive shaft 14 and extending radially outward from the main body 38. The drive system 36 may be configured to move the inner drive shaft 14 axially within the hollow inner plenum 40 of the elongated outer housing 12. The drive system 36 may be formed from a first drive handle 60 and a second drive handle 62 configured such that the first drive handle 60 is movable relative to the second drive handle 62 to move the inner drive shaft 14 axially. The first drive handle 60 may be coupled to a pivot pin 64 such that the first drive handle 60 may pivot about the pivot pin 64. Moving the first drive handle 60 towards the second drive handle 62 may cause the inner drive shaft 14 to move axially toward the distal end 42 of the elongated outer housing 12, which in turn may drive the working head 52. The second drive handle 62 may be rigidly coupled to the main body 38. The first drive handle 60 may be moved towards the second drive handle 62 that may be positioned closer towards the proximal end 20 of the instrument 10 than the first drive handle 62. The first drive handle 60 or the second drive handle 62, or both, may include a finger receptacle 66 configured to receive one or more fingers of a user's hand. The finger receptacle 66 may have any appropriate size and configuration.

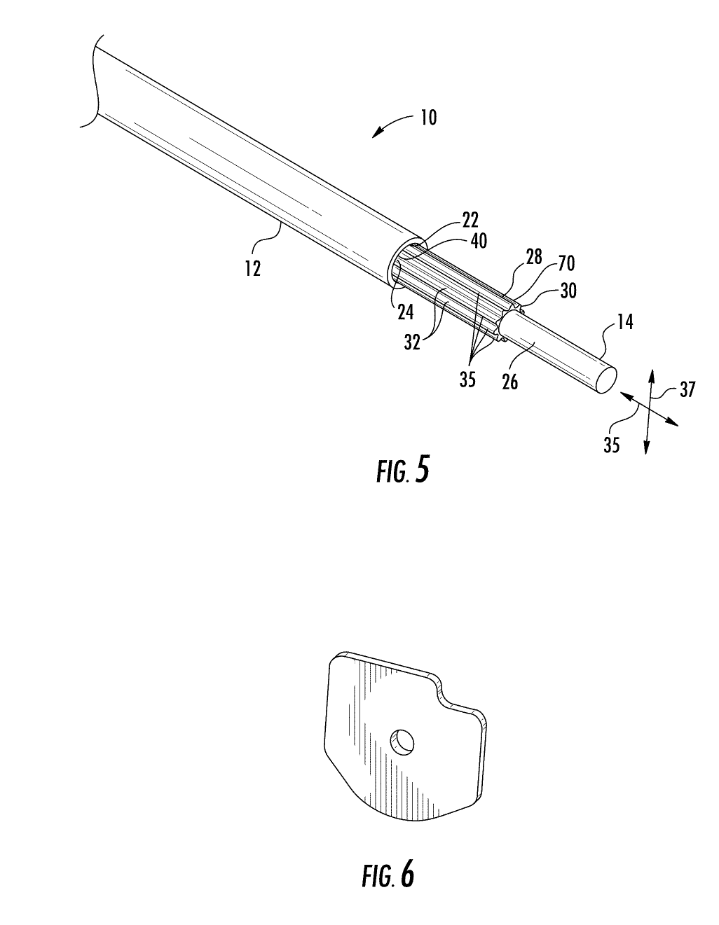

The medical instrument 10 may include an instrument flushing system 16, as shown in FIG. 4, in communication with the hollow cavity 48 within the main body 38 and configured to pass flushing fluid through the hollow cavity 48 within the main body 38 and through the flushing fluid plenum 22 in the hollow inner plenum 40 of the elongated outer housing 12 between the outer surface 26 of the inner drive shaft 14 and the inner surface 24 of the elongated outer housing 12. The instrument flushing system 16 may be configured to supply flushing fluids through the interior aspects of the medical instrument 10 to remove bodily tissue from the medical instrument 10. The instrument flushing system 16 may include one or more ports 18 positioned at a proximal end 20 of the main body 38 of the medical instrument 10. The instrument flushing system 16 may be configured such that the port 18 is axially aligned with the hollow cavity 48 within the main body 38 and the flushing fluid plenum 22 between the elongated outer housing 12 and the inner drive shaft 14. As such, the instrument flushing system 16 is capable of successfully providing flushing action to inner aspects of the medical instrument 10 and to one or more working heads 52 attached to the distal end 54 of the inner drive shaft 14. The port 18 of the instrument flushing system 16 may be a releasable connection, as shown in FIG. 4. The releasable connection may be, but is not limited to being, a LUER-LOK fitting 19. The releasable connection may be a female or male LUER-LOK fitting 19. The LUER-LOK fitting 19 may be releasably attached to the port 18, such as, but not limited to being, threadably attached. The LUER-LOK fitting 19 may include threads 21 on an end 23 of the LUER-LOK fitting 19.

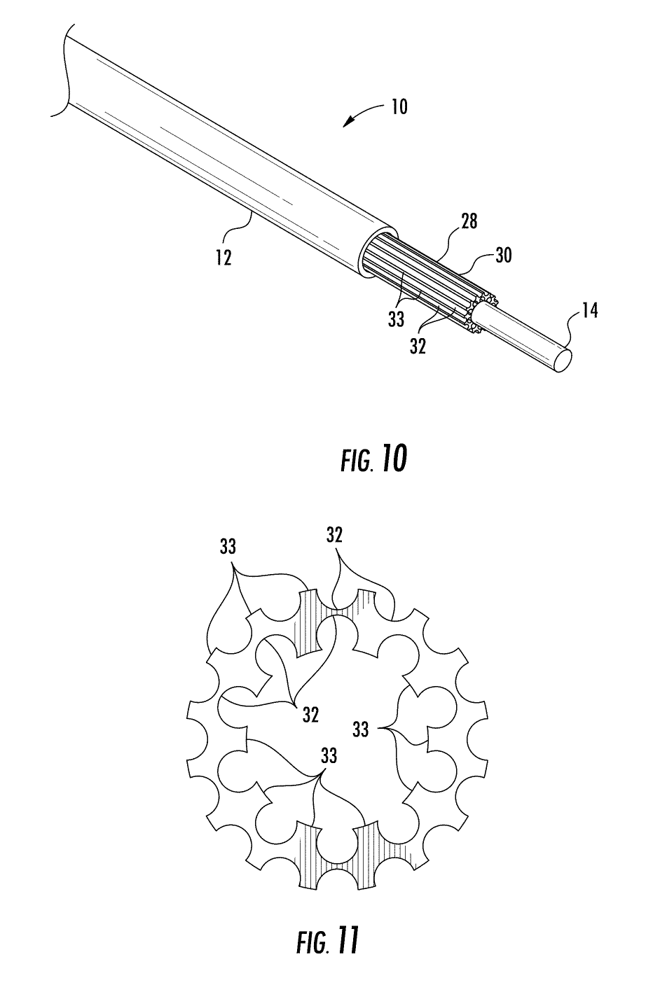

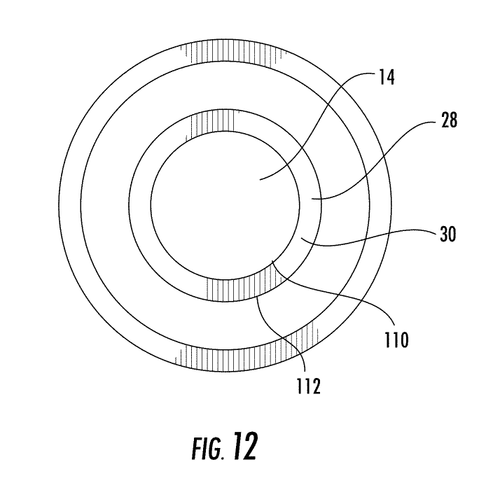

The instrument flushing system 16 may include one or more standoffs 28, as shown in FIGS. 4 and 5, positioned in the flushing fluid plenum 22. The standoffs 28 may be formed from different configurations, as shown, for example, in FIGS. 5, 8 and 9. FIG. 5 discloses a configuration of the standoff 28 in which a plurality of axially extending grooves 32 are formed from a plurality of ridges 33 that extend outwardly in a radial direction 37. FIGS. 8 and 9 discloses a configuration of the standoff 28 in which a plurality of axially extending grooves 32 are formed from a plurality of ridges 33 that extend inwardly in a radial direction 37. FIGS. 10 and 11 disclose a configuration of the standoff 28 in which a plurality of axially extending grooves 32 are formed from a plurality of ridges 33 that extend outwardly in a radial direction 37 and a plurality of axially extending grooves 32 are formed from a plurality of ridges 33 that extend inwardly in a radial direction 37.

The instrument flushing system 16, as shown in FIG. 5, may include one or more standoffs 28 extending between the outer surface 26 of the inner drive shaft 14 and the inner surface 24 of the elongated outer housing 12 to maintain the flushing fluid plenum 22. The standoff 28 need not contact both the outer surface 26 and the inner surface 24 at the same time. The standoff 28 of the instrument flushing system 16 may be formed from a collar 30 extending radially outward from the outer surface 26 of the inner drive shaft 14 whereby the collar 30 may include one or more axially extending grooves 32 therein. The collar 30 may include a plurality of axially extending grooves 32. The axially extending grooves 32 may be formed from a plurality of ridges 33 that extend outwardly in a radial direction 37 and lengthwise in an axial direction 35. The ridges 33 may be separated by a curved surface. The curved grooves 32 may have, but are not limited to having, a semi-circular curved surface. The plurality of axially extending grooves 32 may be aligned with each other, or one or more of the plurality of axially extending grooves 32 may be misaligned with each other. The grooves 32 may have the same depth radially inward from an outer surface 70 of the collar 30, or one or more, or all of the grooves 32 may have different depths into the collar 30. The collar 30 may be an elongated tube extending an entire length of the inner drive shaft 14 or for only a portion of the inner drive shaft 14. The elongated tube may be positioned concentrically about the inner drive shaft 14. The elongated tube may be formed from a material, such as, but not limited to, polytetrafluoroethylene (PTFE), which may be, but is not limited to being, TEFLON.

As shown in FIGS. 8 and 9, the instrument flushing system 16 may include one or more standoffs 28 extending between the inner surface 24 of the elongated outer housing 12 and the outer surface 26 of the inner drive shaft 14 to maintain the flushing fluid plenum 22. The standoff 28 need not contact both the outer surface 26 and the inner surface 24 at the same time. The standoff 28 of the instrument flushing system 16 may be formed from a collar 30 extending radially inward from the inner surface 24 of the elongated outer housing 12 whereby the collar 30 may include one or more axially extending grooves 32 therein. The collar 30 may include a plurality of axially extending grooves 32. The axially extending grooves 32 may be formed from a plurality of ridges 33 that extend inward in a radial direction 37 and lengthwise in an axial direction 35. The ridges 33 may be separated by a curved surface. The curved grooves 32 may have, but are not limited to having, a semi-circular curved surface. The plurality of axially extending grooves 32 may be aligned with each other, or one or more of the plurality of axially extending grooves 32 may be misaligned with each other. The grooves 32 may have the same depth radially inward from an outer surface 70 of the collar 30, or one or more, or all of the grooves 32 may have different depths into the collar 30. The collar 30 may be an elongated tube extending an entire length of the inner drive shaft 14 or for only a portion of the inner drive shaft 14. The elongated tube may be positioned concentrically about the inner drive shaft 14. The elongated tube may be formed from a material, such as, but not limited to, PTFE, which may be, but is not limited to being, TEFLON.

As shown in FIGS. 10 and 11, the instrument flushing system 16 may include one or more standoffs 28 extending between the inner surface 24 of the elongated outer housing 12 and the outer surface 26 of the inner drive shaft 14 to maintain the flushing fluid plenum 22. The standoff 28 need not contact both the outer surface 26 and the inner surface 24 at the same time. The standoff 28 of the instrument flushing system 16 may be formed from a collar 30 positioned in the hollow inner plenum 40. The collar 30 may include a plurality of ridges 33 extending inwardly and a plurality of ridges 33 extending outwardly. In particular, the collar 30 may include a plurality of axially extending grooves 32 are formed from a plurality of ridges 33 that extend outwardly in a radial direction 37 and a plurality of axially extending grooves 32 are formed from a plurality of ridges 33 that extend inwardly in a radial direction 37. The axially extending grooves 32 may be formed from a plurality of ridges 33 that extend inward in a radial direction 37 and lengthwise in an axial direction 35. The ridges 33 may be separated by a curved surface. The curved grooves 32 may have, but are not limited to having, a semi-circular curved surface. The plurality of axially extending grooves 32 may be aligned with each other, or one or more of the plurality of axially extending grooves 32 may be misaligned with each other. The grooves 32 may have the same depth radially inward from an outer surface 70 of the collar 30, or one or more, or all of the grooves 32 may have different depths into the collar 30. The collar 30 may be an elongated tube extending an entire length of the inner drive shaft 14 or for only a portion of the inner drive shaft 14. The elongated tube may be positioned concentrically about the inner drive shaft 14. The elongated tube may be formed from a material, such as, but not limited to, PTFE, which may be, but is not limited to being, TEFLON.

As shown in FIG. 12, the instrument flushing system 16 may include inner drive shaft 14 positioned within the elongated outer housing 12 and extending into the hollow cavity 48 within the main body 38. The inner drive shaft 14 may be sized such that a flushing fluid plenum 22 exists in the hollow inner plenum 40 of the elongated outer housing 12 between an outer surface 26 of the inner drive shaft 14 and an inner surface 24 of the elongated outer housing 12 when the inner drive shaft 14 is positioned within the outer housing 12. The instrument flushing system 16 may include a standoff 28 formed from a collar 30. The collar 30 may include a smooth inner surface 110 and a smooth outer surface 112. The collar 30 may extend for all or a portion of a length of the inner drive shaft 14.

The instrument flushing system 16 may be configured to prevent or substantially limit leakage of a flushing fluid from the medical instrument 10 while a flushing fluid, such as, but not limited to, water, is passed through the instrument flushing system 16 within the medical instrument 10. The instrument flushing system 16 may include one or more seals 34, as shown in FIGS. 3, 4 and 6, to prevent leakage of the flushing fluid from the main body 38 where the first drive handle 60 extends from the main body 38. The seal 34 may be formed form any appropriate configuration, size and material or materials, such as, but not limited to polytetrafluoroethylene (PTFE), which may be, but is not limited to being, TEFLON. The seal 34, as shown in FIGS. 3, 4 and 6, may be formed from a first side 72 and a second side 74. The seal 34 may be formed from any appropriate manufacturing method. In at least one embodiment, the seal 34 may be formed from a stamping process in which the first and section sides 72, 74 may be stamped from a sheet of material. The first and second sides 72, 74 may include first and second sidewalls 76, 78 separated by a void sized to receive the first drive handle 60. When the first and second sides 72, 74 are positioned within the main body 38, the first and second sidewalls 76, 78 may contact each other to assist in sealing the hollow cavity 48 and to form a single opening 80 through which the first drive handle 60 extends. The seal 34 may be configured such that the first and second sides 72, 74 press against the first drive handle 60 tightly but not too tight that the friction prevents the first drive handle 60 from being moved relative to the main body 38. The first and second sides 72, 74 may have linear surfaces.

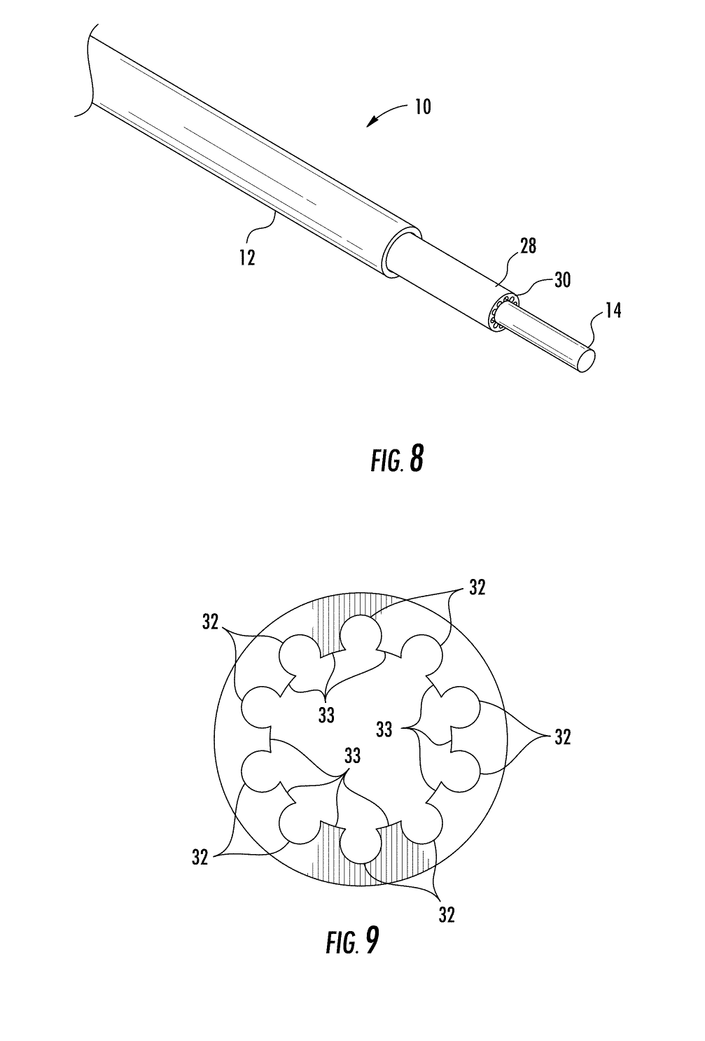

A method of cleaning a medical instrument at 90, as shown in FIG. 7, may include cleaning a medical instrument 10, as previously set forth. In particular, the method 90 may include providing fluid to the medical instrument at 92. The medical instrument 10 may be any appropriate configuration, such as, but not limited to, an elongated outer housing 12 with a hollow inner plenum 40, whereby a distal end 42 of the elongated outer housing 12 includes an outer shaft distal opening 44. The medical instrument 10 may also include a main body 38 extending proximally from a proximal end 46 of the elongated outer housing 12. The medical instrument 10 may also include an inner drive shaft 14 positioned within the elongated outer housing 12 and extending into a hollow cavity 48 within the main body 38, whereby the inner drive shaft 14 is sized such that a flushing fluid plenum 22 exists in the hollow inner plenum 40 of the elongated outer housing 12 between an outer surface 26 of the inner drive shaft 14 and an inner surface 24 of the elongated outer housing 12. The medical instrument 10 may also include a drive system 36 in communication with the inner drive shaft 14 and extending radially outward from the main body 38, whereby the drive system 36 may be configured to move the inner drive shaft 14 axially within the hollow inner plenum 40 of the elongated outer housing 12. The medical instrument 10 may also include an instrument flushing system 16 in communication with the hollow cavity 48 within the main body 38 and configured to pass flushing fluid through the hollow cavity 48 within the main body 38 and through the flushing fluid plenum 22 in the hollow inner plenum 40 of the elongated outer housing 12 between the outer surface 26 of the inner drive shaft 14 and the inner surface 24 of the elongated outer housing 12.

The method 90 may also include enabling flush fluid to flow at 94 into the instrument flushing system 16. The step of enabling flush fluid to flow at 94 into the instrument flushing system 16 may include opening a valve (not shown), starting a pump (not shown) in fluid communication with the instrument flushing system 16, depressing a plunger within a syringe 25 in fluid communication with the instrument flushing system 16 and the like. Once flushing fluid is delivered to the instrument flushing system 16, such as into at least one port 18 of the instrument flushing system 16 at a proximal end 20 of the main body 38 of the medical instrument 10, the fluid fills the hollow cavity 48 within the main body 38 of the medical instrument 10. The flushing fluid flows from the hollow cavity 48 within the main body 38 of the medical instrument 10 and into the flushing fluid plenum 22 within the elongated outer housing 12. When flowing through the flushing fluid plenum 22, the flushing fluid will pass through one or more axially extending grooves 32 in a standoff 28, such as, but not limited to, the collar 30. The standoff 28, such as, but not limited to, the collar 30 positions the inner drive shaft 14 within the elongated outer housing 12 to maintain the flushing fluid plenum 22 to prevent inner drive shaft 14 from blocking the flushing fluid plenum 22. The flushing fluid may be exhausted from the flushing fluid plenum 22 and carry with it tissue and blood found within the flushing fluid plenum 22 and at the one more working heads 52.

The step of providing fluid to the medical instrument 10 at 92 may include providing fluid to the medical instrument 10 via at least one port 18 of the instrument flushing system 16 at a proximal end 20 of the main body 38 of the medical instrument 10. The step of providing fluid to the medical instrument 10 at 92 may also include attaching the medical instrument 10 to a fluid source, which may be, but is not limited to being, a syringe, a tube connected to a pressurized fluid source and the like. The step of providing fluid to the medical instrument 10 at 92 may include providing fluid to the medical instrument 10 via a releasable connection at least one port 18. The step of providing fluid to the medical instrument 10 at 92 may include providing fluid to the medical instrument 10 including at least one standoff 28 positioned in the flushing fluid plenum 22 and extending between the outer surface 26 of the inner drive shaft 14 and the inner surface 24 of the elongated outer housing 12 to maintain the flushing fluid plenum 22. The step of providing fluid to the medical instrument 10 at 92 may also include providing fluid to the medical instrument 10 wherein the standoff 28 of the instrument flushing system 16 is formed from a collar 30 extending radially outward from the outer surface 26 of the inner drive shaft 14, whereby the collar 30 includes one or more axially extending grooves 32 therein. The step of providing fluid to the medical instrument 10 at 92 may include providing fluid to the medical instrument 10 wherein the standoff 28 is formed from a collar 30 extending radially outward from the outer surface 26 of the inner drive shaft 14, wherein the collar 30 includes a plurality of axially extending grooves 32 positioned in a radially outer surface 70 of the collar 30. The step of providing fluid to the medical instrument 10 at 92 may include providing fluid to the medical instrument 10 wherein the collar 30 includes a plurality of axially extending grooves 32 that are aligned with each other. The step of providing fluid to the medical instrument 10 at 92 may include providing fluid to the medical instrument 10 wherein the drive system 36 is formed from a first drive handle 60 and a second drive handle 62 configured to be movable relative to each other to move the inner drive shaft 14 axially and the instrument flushing system 16 further includes at least one seal 34 configured to seal the hollow cavity 48 within the main body 38 where the first drive handle 60 extends radially outward from the main body 38.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A medical instrument, comprising:
an elongated outer housing with a hollow inner plenum, wherein a distal end of the elongated outer housing includes an outer shaft distal opening;
a main body extending proximally from a proximal end of the elongated outer housing;
an inner drive shaft positioned within the elongated outer housing and extending into a hollow cavity within the main body, wherein the inner drive shaft is sized such that a flushing fluid plenum exists in the hollow inner plenum of the elongated outer housing between an outer surface of the inner drive shaft and an inner surface of the elongated outer housing;
a drive system in communication with the inner drive shaft and extending radially outward from the main body, wherein the drive system is configured to move the inner drive shaft axially within the hollow inner plenum of the elongated outer housing; and
an instrument flushing system in communication with the hollow cavity within the main body and configured to pass flushing fluid through the hollow cavity within the main body and through the flushing fluid plenum in the hollow inner plenum of the elongated outer housing between the outer surface of the inner drive shaft and the inner surface of the elongated outer housing.

2. The medical instrument of claim 1, wherein the instrument flushing system further comprises at least one port at a proximal end of the main body of the medical instrument.

3. The medical instrument of claim 2, wherein the at least one port of the instrument flushing system is a releasable connection.

4. The medical instrument of claim 1, wherein the instrument flushing system further comprises at least one standoff positioned in the flushing fluid plenum and extending between the outer surface of the inner drive shaft and the inner surface of the elongated outer housing to maintain the flushing fluid plenum.

5. The medical instrument of claim 4, wherein the at least one standoff of the instrument flushing system is formed from a collar extending radially outward from the outer surface of the inner drive shaft, wherein the collar includes at least one axially extending groove therein.

6. The medical instrument of claim 5, wherein the collar includes a plurality of axially extending grooves.

7. The medical instrument of claim 6, wherein the plurality of axially extending grooves are aligned with each other and are positioned in a radially outer surface of the collar.

8. The medical instrument of claim 5, wherein the collar is an elongated tube positioned concentrically about the inner drive shaft.

9. The medical instrument of claim 4, wherein the at least one standoff of the instrument flushing system is formed from a collar extending radially inward from the inner surface of the elongated outer housing, wherein the collar includes at least one axially extending groove therein.

10. The medical instrument of claim 9, wherein the collar further comprises at least one axially extending groove extending radially outward toward the collar toward the inner surface of the elongated outer housing.

11. The medical instrument of claim 1, wherein the drive system is formed from a first drive handle and a second drive handle configured to be movable relative to each other to move the inner drive shaft axially.

12. The medical instrument of claim 11, wherein the instrument flushing system further comprises at least one seal configured to seal the hollow cavity within the main body where the first and second drive handles extend radially outward from the main body.

13. A method of cleaning a medical instrument, comprising:
providing fluid to the medical instrument, comprising:
an elongated outer housing with a hollow inner plenum, wherein a distal end of the elongated outer housing includes an outer shaft distal opening;
a main body extending proximally from a proximal end of the elongated outer housing;
an inner drive shaft positioned within the elongated outer housing and extending into a hollow cavity within the main body, wherein the inner drive shaft is sized such that a flushing fluid plenum exists in the hollow inner plenum of the elongated outer housing between an outer surface of the inner drive shaft and an inner surface of the elongated outer housing;
a drive system in communication with the inner drive shaft and extending radially outward from the main body, wherein the drive system is configured to move the inner drive shaft axially within the hollow inner plenum of the elongated outer housing; and
an instrument flushing system in communication with the hollow cavity within the main body and configured to pass flushing fluid through the hollow cavity within the main body and through the flushing fluid plenum in the hollow inner plenum of the elongated outer housing between the outer surface of the inner drive shaft and the inner surface of the elongated outer housing; and
enabling flush fluid to flow into the instrument flushing system.

14. The method of cleaning a medical instrument of claim 13, wherein providing fluid to the medical instrument comprises providing fluid to the medical instrument via at least one port of the instrument flushing system at a proximal end of the main body of the medical instrument.

15. The method of cleaning a medical instrument of claim 14, wherein providing fluid to the medical instrument via the at least one port of the instrument flushing system comprises providing fluid to the medical instrument via a releasable connection at the at least one port of the instrument flushing system at a proximal end of the main body of the medical instrument.

16. The method of cleaning a medical instrument of claim 13, wherein providing fluid to the medical instrument comprises providing fluid to the medical instrument wherein the instrument flushing system further comprises at least one standoff positioned in the flushing fluid plenum and extending between the outer surface of the inner drive shaft and the inner surface of the elongated outer housing to maintain the flushing fluid plenum.

17. The method of cleaning a medical instrument of claim 16, wherein providing fluid to the medical instrument comprises providing fluid to the medical instrument wherein the at least one standoff of the instrument flushing system is formed from a collar extending radially outward from the outer surface of the inner drive shaft, wherein the collar includes at least one axially extending groove therein.

18. The method of cleaning a medical instrument of claim 17, wherein providing fluid to the medical instrument comprises providing fluid to the medical instrument wherein the at least one standoff of the instrument flushing system is formed from a collar extending radially outward from the outer surface of the inner drive shaft, wherein the collar includes a plurality of axially extending grooves positioned in a radially outer surface of the collar.

19. The method of cleaning a medical instrument of claim 18, wherein providing fluid to the medical instrument comprises providing fluid to the medical instrument wherein the at least one standoff of the instrument flushing system is formed from a collar extending radially outward from the outer surface of the inner drive shaft with a plurality of axially extending grooves positioned in a radially outer surface of the collar wherein the plurality of axially extending grooves are aligned with each other.

20. The method of cleaning a medical instrument of claim 13, wherein providing fluid to the medical instrument comprises providing fluid to the medical instrument wherein the drive system is formed from a first drive handle and a second drive handle configured to be movable relative to each other to move the inner drive shaft axially and the instrument flushing system further comprises at least one seal configured to seal the hollow cavity within the main body where the first and second drive handles extend radially outward from the main body.
